(19) United States
(12) Patent Application Publication
(10) Pub. No.: US 2018/0325414 A1
(43) Pub. Date: Nov. 15, 2018

(54) ELECTRO-MAGNETO VOLUME TOMOGRAPHY SYSTEM AND METHODOLOGY FOR NON-INVASIVE VOLUME TOMOGRAPHY

(71) Applicant: Tech4Imaging LLC, Columbus, OH (US)

(72) Inventors: Qussai Marashdeh, Columbus, OH (US); Christopher Zuccarelli, Columbus, OH (US)

(21) Appl. No.: 15/593,643

(22) Filed: May 12, 2017

(51) Int. Cl. A61B 5/053; G06T 11/00

(52) U.S. Cl. CPC A61B 5/0536; G01N 27/226; G06T 11/008

(57) ABSTRACT

A system and method capable of performing multiple types of non-invasive tomographic techniques. The system is capable, via electronic control, of detecting and imaging materials within a volume using electrical capacitance, displacement phase current, magnetic inductance, and magnetic pressure sensing. The system is also able to control the amplitude, phase, and frequency of individual electrode excitation to increase imaging resolution and phase detection. This allows many dimensions of non-invasive data to be captured without the need for multiple instruments or moving parts, at a high data capture rate.

### BACKGROUND OF THE INVENTIVE FIELD

There has been much work and research in the fields of noninvasive volumetric imaging in the past century. Various phenomena and physics principals have been used as methods to look inside of objects and spaces without physically opening them. The most well-known and widely-used methods include X-ray and magnetic resonance imaging (MRI), which have numerous applications, especially in the medical field. Other types of noninvasive tomography include ultrasonic, beam diffraction (for imaging within metal sheets), thermal imaging, and light diffraction.

Electrical Capacitance Tomography (ECT) involves the use of an array of flat conducting plates placed around a region of interest. A low power, low frequency electric field is sent out from one of these plates and detected at another plate to measure the capacitance between the plates. This capacitance value changes as the material type or distribution in between these plates changes. By using enough unique plate pairs, ECT can provide reliable quantitative data about the distribution and flow pattern of the material or materials within its sensing region. This type of tomography is advantageous when studying mainly insulative materials.

ECT is the reconstruction of material concentrations of dielectric physical properties in the imaging domain by inversion of capacitance data from a capacitance sensor. Electrical Capacitance Volume Tomography or ECVT is the direct 3D reconstruction of volume concentrations or physical properties in the imaging domain utilizing 3D features in the ECVT sensor design. ECVT technology is described in U.S. Pat. No. 8,614,707 to Warsito et al. which is hereby incorporated by reference.

Electrical Capacitance Volume Tomography (ECVT) is a non-invasive imaging modality. Its applications span an array of industries. Most notably, ECVT is applicable to multiphase flow applications commonly employed in many industrial processes. ECVT is often the technology of choice due to its advantages of high imaging speed, scalability to different process vessels, flexibility, and safety. In ECVT, sensor plates are distributed around the circumference of the column, object or vessel under interrogation. The number of sensor plates may be increased to acquire more capacitance data. However, increasing the number of sensor plates reduces the area of each sensor plate accordingly. A limit exists on the minimum area of a sensor plate for a given column diameter, thus limiting the maximum number of plates that can be used in an ECVT sensor. This limit is dictated by the minimum signal-to-noise ratio requirement of the data acquisition system. Since ECVT technology is based on recording changes in capacitance measurements induced by changes in dielectric distribution (i.e., phase distribution), and the capacitance level of a particular sensor plate combination is directly proportional to the area of the plates, minimum signal levels are needed to provide sufficiently accurate measurements. These considerations dictate the required minimum sensor plate dimensions. This limitation on the minimum size of the sensor plates, while increasing the number of available sensor plates in an ECVT sensor, is one of the main hurdles in achieving a high resolution imaging system.

To overcome this challenge, the concept of Adaptive Electrical Capacitance Volume Tomography (AECVT) was recently created, whereby the number of independent capacitance measurements is increased through the use of reconfigurable synthetic sensor plates composed of many smaller sensor plates (constitutive segments). These synthetic sensor plates maintain the minimum area for a given signal-to-noise ratio (SNR) and acquisition speed requirements while allowing for many different combinations of (synthetic) sensor plates in forming a sensor plate pair.

An ECVT system is generally made up of a sensor, sensor electronics and a computer system for reconstruction of the image sensed by the sensor. An ECVT sensor is generally comprised of n electrodes or plates placed around a region of interest, in one embodiment providing n(n−1)/2 independent mutual capacitance measurements which are used for image reconstruction. Image reconstruction is performed by collecting capacitance data from the electrodes placed around the wall outside the vessel.

Adaptive Electrical Capacitance Volume Tomography (AECVT) provides higher resolution volume imaging of capacitance sensors based on different levels of activation levels on sensor plate segments. In AECVT systems, electrodes are comprised of an array of smaller capacitance segments that may be individually addressed. For example, each segment may be activated with different amplitudes, phase shifts, or frequency to provide the desired sensitivity matrix distribution. The sensor electronics of the present invention is designed to detect and measure the capacitance for the adaptive ECVT sensor of the present invention. For example, the difference in electrical energy stored in the adaptive ECVT sensor would be measured between an empty state and a state where an object is introduced into the imaging domain (e.g., between the electrodes). In a preferred embodiment of the invention, the term “adaptive” means the ability to provide selective or high resolution control through the application of voltage or voltage distributions to a plate having an array of capacitance segments. The change in overall energy of the system due to the introduction of a dielectric material in the imaging domain is used to calculate the change in capacitance related to the dielectric material. The change in capacitance can be calculated from the change in stored energy. Sensor electronics can also be designed by placing individual segment circuits in parallel yielding a summation of currents representing total capacitance between segments under interrogation. By individually addressing the capacitance segments of the electrodes of the present invention, electric field distribution inside the imaging domain can be controlled to provide the desired sensitivity matrix, focus the electric field, and increase overall resolution of reconstructed images. Voltage distribution can also be achieved by using a conventional measuring circuit with a sensor that distributes voltages through a voltage divider.

In AECVT systems, a capacitance measurement circuit is connected to an electrode (detecting or receiving electrode) of the adaptive sensor so that a capacitance measurement can be obtained for the selected source and detecting electrodes. The capacitors Cx1-Cxn of the sensor represent the n number of capacitance segments of the selected source electrode and the detecting electrode. Each capacitance segment of the electrodes can be individually addressed by separated voltage sources. These voltage sources are used for regulating the voltage levels and phase shifts on the capacitance segments of each of the electrodes on the adaptive sensor. The voltage across each of the capacitor segments (Vxn) is the combination of the voltage source Vi and the voltage sources connected to each capacitor segment (Vn). Accordingly, the measured Vo can be used to calculate each of the equivalent capacitance (Cxn) of the capacitance segments of the activated electrode. The associated formula is for Cxn=Cx1=Cx2 . . . =Cxi. For segments with different capacitance values, the equivalent capacitance is calculated using the formula:

$${V}_{0} = ( \frac{j ω {R}_{f}}{1 + j ω {C}_{f} {R}_{f}} ) ( {Σ}_{i = 1}^{n} {V}_{xi} {C}_{xi} )$$

As discussed, in one embodiment, n(n−1)/2 independent mutual capacitance measurements are measured and used for image reconstruction. For example, the capacitance between each of the electrodes of the sensor are measured in turn and image reconstruction is performed using this capacitance data. In other words, capacitance measurements are obtained from every pair or electrode combination of the sensor, in turn, to be used in image reconstruction. It is appreciated that the voltage sources herein discussed may be connected to the capacitance segments of each of the electrodes of the sensor array using known switch technologies. Using switches, the system can selectively choose which electrodes to activate by connecting the voltage sources to the selected electrodes through the switches. In another embodiment, switching or multiplexing circuit elements can be used to connect the appropriate voltage sources to each of the capacitance segments of the selected electrode allowing various elements to be selectively connected to each capacitance segment depending on the focus and sensitivity desired. For example, voltage sources of greater amplitude may be switched or connected to the capacitance segments in the center of the electrode or imaging domain so as to focus the measurements towards the center of the electrode or imaging domain.

In an alternate embodiment, instead of using different amplitudes, different frequencies may be used to activate electrode segments enabling concurrent measurements of different capacitance values introduced by electric field beams of different frequencies. In yet another alternate embodiment, different phase shifts may be used to activate electrode segments enabling steering of the electric field inside the imaging domain. The measured change in output voltage can be used to calculate the change in capacitance levels between the capacitance segments which are then used to reconstruct volume images of objects or materials between the sensors. AECVT is described in U.S. Patent Application Publication US2013/0085365 A1 and U.S. Pat. No. 9,259,168 to Marashdeh et al. which are hereby incorporated by reference.

A new reconstruction methodology of AECVT known as the Space Adaptive Reconstruction Technique (SART) is designed to utilize the flexibility of the AECVT technique in such a way that the imaging domain is divided into several regions where each region's permittivity distribution is reconstructed independently, based on “a priori” information about other region's calculated permittivity distributions. The algorithm iteratively reconstructs the spatial permittivity distribution of each separate region in the imaging domain until convergence is achieved. This process may also involve staggered iterative methods where each region is reconstructed iteratively and the independent regions are then combined into one image through another iterative optimization process. The basic principle behind this new reconstruction algorithm is that the fundamental resolution provided by the segment plates decreases monotonically from the periphery of the imaging domain close to the segment plates toward the center of the imaging domain far from the segment plates, due to the Laplace nature of interrogating the quasi-static electric field. Therefore, in electrical capacitance tomography applications, the field lines that penetrate into the middle of the imaging domain are always weaker and more spread out compared to those closer to the sensor plates. The spatial sensitivity of any given capacitance sensor plates (to permittivity variations) is much greater at points in close vicinity to it when compared to points farther away from it. This causes the image resolution to progressively degrade at regions further away from the sensor plates.

In ECT, ECVT, or AECVT, the capacitance measurement between sensor plates is also related to the effective dielectric content between that plate pair. The SART method can be extended to all measurements of ECT, ECVT, or AECVT sensors, thus providing a high resolution visual representation of each phase through image reconstruction. These previous ECVT systems incorporate data acquisition system that increase imaging resolution through sensing capacitances from 3D conventional and adaptive capacitance sensors. Data acquisition systems are also described in U.S. patent application Ser. No. 14/191,574 (Publication No. US-2014-0365152-A1) which is hereby incorporated by reference.

Electrical capacitance sensors are used for non-invasive imaging by distributing the electric field inside the imaging domain in 3D. ECVT sensors enable sensitivity variation in the imaging domain that can utilize different plate shapes and distributions to target a volume for imaging.

A data acquisition system operating at multiple frequencies is required for phase decomposition. Capacitances can be measured at different frequencies successively or simultaneously. In the former approach, the data acquisition speed of capacitance values at different frequencies should be higher than flow speed. In the latter, a synchronous demodulator is used to isolate each capacitance value related to each frequency. Using both measuring schemes, the difference between measured capacitances (successive or simultaneous) is used to isolate the change in effective dielectric constant for multi-phase flow decomposition. Multi-phase flow decomposition using this technique is described in U.S. patent application Ser. No. 15/138,751 to Marashdeh et al. which is hereby incorporated by reference.

Electrical capacitance sensors are used for non-invasive imaging by distributing the electric field inside the imaging domain in 3D. ECVT sensors enable sensitivity variation in the imaging domain that can utilize different plate shapes and distributions to target a volume for imaging. They exhibit flexibility for fitting around different sizes and geometries and are scalable to different sizes. Capacitance sensors so far have been focused on being passively applied around a geometry. In such arrangements, the capacitance plates are designed to fit around the targeted geometry and the sensor shape is recorded for image reconstruction purposes. In a recent invention, capacitance sensors are designed with a smart feature that enables the sensor to detect and quantify the geometry it is placed around. Capacitance sensors in this case are developed from flexible materials that can be used for imaging volumes of different shapes or sizes. The smart capacitance sensor is able to detect the shape and size of the volume it is placed around formulate a sensitivity matrix for such volume, acquire capacitance measurements, and provide reconstructed images. Each pair of inner geometry sensor plates detect a capacitance signal that has information on how much the sensor stretched in that region. The smart feature using this technique is described in U.S. patent application Ser. No. 15/152,031 to Marashdeh et al. which is hereby incorporated by reference.

Non-linearity is often a problem in relating the material distribution and permittivity of the sensing region to the signal received by the capacitance sensor, especially in applications of higher permittivity materials such as water. This high permittivity, or dielectric constant, amplifies the non-linearity in the image reconstruction problem which can further complicate the process of extracting images and other information from the measured signal.

A recently-discovered type of tomography is Displacement Current Volume Tomography, or DCPT. Also noninvasive, this relates to a system and process to obtain a linear relationship between mutual displacement current from the sensor (output current of the measuring electrode terminals) and the area (or volume) of an object to be imaged in the imaging domain. This new system uses capacitance sensors and utilizes the phase of the measured current, in addition to the amplitude, to reconstruct an image. This new system is named Displacement Current Phase Tomography (DCPT). Similar to ECVT, DCPT is a low-cost imaging modality with the potential of being very useful to image two or three phase flow systems where there is a high contrast in the dielectric constant or where the material being imaged is lossy due to presence of electric conductivity or dielectric loss.

In conventional ECVT, the current amplitude across the capacitor plates is measured, which is then used to calculate the mutual capacitance between a plate pair and then used to reconstruct a 3D image. When objects in the imaging domain are lossy (i.e., having electric conductivity or dielectric loss), there is additional information contained in the phase of the currents that can also be used for image reconstruction. The change in the current phase is nearly linear with the volume fraction for many lossy materials. In ECVT, the sensitivity map is based on the current phase information. Traditionally the sensitivity map joins the capacitance (current amplitude) to the permittivity distribution, and thus the material distribution, in the imaging domain through a linear approximation. Because the current phase is also linearly related to material distribution, it can provide an alternative imaging process using a similar linearized sensitivity matrix. Comparable to conventional ECVT, the phase sensitivity matrix which is calculated for all pixel locations is used in conjunction with the measurement of the current phase to reconstruct the volumetric image of the material distribution (spatial distribution of the conductivity or dielectric loss) in the sensing region.

The phase information can also be used to deduce velocity of a material moving through the sensing region. There is a relaxation time, dependent on material conductivity and dielectric properties, between when the electric field is applied to a material and when the material fully responds in dielectric polarization. The material first enters the sensing region and is them polarized by the electric field. As the material exits the sensing region, the material is still polarized and relaxes according to its relaxation time constant. This relaxation occurring outside the sensor plate's effective zone produces a change in the phase of the measured current, and thus any changes in the measured displacement current phase of the sensor when material is through the sensing region can be directly related to the velocity of the material. This observation is useful when dealing with single phase flows where the effective capacitance does not change, which typically renders cross-correlation methods for calculating velocity useless under conventional ECVT methods. DCPT solves this ECVT problem by relating the measured current phase directly to the velocity of the moving material when the effective dielectric constant inside the sensing region does not change. DCPT technology is described in U.S. patent application Ser. No. 15/265,565 which is hereby incorporated by reference.

Magnetic Inductance Tomography (MIT) employs inductance to noninvasively monitor a sensing region. Coils of conductive wire are placed around a region of interest. An alternating current is run through one of these coils to produce a magnetic field pointing away from the coil, into the sensing region. This alternating magnetic field induces an alternating current within another coil, which can be measured. The measured current varies as the material in between the coils changes. By using enough unique coil pairs, MIT can provide reliable quantitative data about the distribution and flow pattern of the material or materials within its sensing region. This type of tomography is advantageous when studying mainly conductive materials.

A newly discovered noninvasive detection technique involves the use of DC magnetic fields to detect metallic objects. Magnetic Press Sensing (MaPS) involves the use of pressure sensors, or strain gauges, to determine the amount and location of ferromagnetic and ferromagnetic objects. Permanent magnets or electrically-activated magnets are placed around a region of interest. Strain gauges are then placed on the magnets to measure changes in forces on the magnets. As magnetic material moves through the sensing region, attractive and repulsive forces on each magnet varies and is detected via the strain gauges. These strains are translated into information about the presence of magnetic material within the sensing region. This type of noninvasive measurement is advantageous when detecting magnetic materials. Measurements using this technique is described in U.S. patent application Ser. No. 15/452,023 to Marashdeh et al. which is hereby incorporated by reference.

These measurement techniques, like any other type of measurement, have their own advantages and disadvantages. Each is more capable in certain situations and less capable in others. Different measurement techniques can be used to measure different materials. It is for this reason that instruments that need to detect and image multiple materials can be quite large, slow to operate, and cumbersome. Multiple instruments, each employing its own unique measurement technique, are combined into a single unit, which can become quite complex and confusing to handle. This is true of most modern-day offerings in multi-phase flow meters (MPFMs), which attempt to measure, in real time, the flow of gases, oil, and water moving through oil fields and pipes.

The present invention serves to combine the functionality of multiple aforementioned noninvasive sensing techniques into an integrated unit in a unique fashion not previously known. This instrument can employ all or some of the following measurement techniques: Electrical Capacitance Tomography, Electrical Capacitance Volume Tomography, Adaptive Electrical Capacitance Volume Tomography, Displacement Current Volume Tomography, Magnetic Pressure Sensing, and Magnetic Inductance Tomography. The instrument can control, via electronics, the type of measurement it records at a high speed, without requiring moving parts. All of these measurement techniques are performed with the single sensor of the present invention, thus keeping the main instrument smaller, more efficient, more accurate, and more flexible. As the instrument and methodology of this noninvasive imaging system revolves around electric and magnetic phenomena, the invention is deemed Electro-Magneto Volume Tomography (EMVT).

### SUMMARY OF THE INVENTION

The present invention relates to a system comprised of a single sensor with accompanying electronics for obtaining multiple dimensions of measurements about a scanned or imaged region, with no moving parts. The system can control:

Excitation amplitude;

Excitation phase shift;

Excitation frequency;

While measuring:

Capacitance amplitude;

Displacement current phase shift;

Inductance amplitude;

Inductance current phase shift;

Magnetic force on electrodes.

In the preferred embodiment of the invention, the sensor portion of the instrument is an electrically-shielded group of electrodes. In one embodiment, the system electrodes are arranged along a surface that encloses a volume of interest. In another embodiment, these electrodes are aligned in a plane and used to image a space in front of the plane. The body which houses these electrodes can be rigid or flexible. In the case of a flexible body, each electrode may be paired with a location sensor that allows hardware and software to automatically determine the shape and volume of the sensor. This allows to real-time adaptation of tomography image reconstruction based on the sensor's current viewing region.

Each electrode is designed and wired so to be able to act as different devices when activated in different ways. By exciting the electrode with an alternating voltage, the electrode acts as a capacitance plate and so can be used to employ Electrical Capacitance Volume Tomography (ECVT) techniques to image a volume. This function is primarily used to detect and image dielectric and low conductive materials.

By passing an alternating current through the electrode, the electrode acts as an inductor and so can be used to employ Magnetic Induction Tomography (MIT) techniques to image a volume. This function is primarily used to detect and image ferromagnetic conductive materials.

By passing a large amount of current though the electrode, the electrode acts as a magnet and is attracted to or repelled by any magnetic material inside the sensing region, and is subjected to an attractive or repulsive force. The magnitude of this force is measured by a strain gauge placed against the electrode. This function is primarily used to detect and image magnetic materials.

One embodiment of the electrode is a non-magnetic conductor, coiled into a single layer or multiple layers. The number of layers controls the magnetic field strength and the surface area of the sensor controls its capacitance operation. This coil is tightly-wound so to maximize surface area and the number of turns in the coil. The electrode is excited with voltage for capacitance operation at one frequency. The electrode is excited with a current to be used as an inductor coil or magnet at a different frequency.

The width of this conductor can be increased to increase its current-carrying capacity and its capacitor capacity. The depth of this conductor can be increased to increase its current-carrying capacity while keeping the same planar configuration. The number of turns in the conductor coil can be increased to increase its inductance.

A ferromagnetic or ferromagnetic material can be placed in the center of this electrode to enhance its ability to serve as an inductor coil or magnet. In an alternate embodiment, the electrode coils can be coiled in a non-concentric configuration so to redirect magnetic field strength in a certain direction when the electrode is in inductance mode.

A data acquisition and control system of present invention is adapted to activate the various states of measurement via the direction of electrical signals to the sensor. The system is adapted and configured, to control the excitation of the sensor electrodes by altering the magnitude, AC phase, and frequency of the electric waves being sent to the electrodes.

It is also possible to activate multiple electrodes at once, each with its own particular magnitude, AC phase, and frequency to more accurately control the electric and magnetic fields within the sensing region. The receiver electrodes can also be chosen in such a manner, so spatial resolution of the sensing region can be more finely controlled. It is also possible, via proper modulation and demodulation, to send and receive multiple frequencies of waveforms at the same time, thus increasing the amount of data gained from the system with no additional time cost or moving parts.

In one embodiment of the invention, the invention is comprised of: a three-dimensional capacitance sensor device comprising a plurality of electrodes for placement around the vessel or the object, wherein the three-dimensional capacitance sensor device is adapted to provide electric field distribution and sensor sensitivity in three geometric dimensions; an activation circuit for activating the three-dimensional capacitance sensor with an activation signal; wherein the activation circuit is adapted to vary the activation signal by amplitude, phase, and frequency; a data acquisition circuit in communication with the three-dimensional capacitance sensor device for receiving output signals from the three-dimensional capacitance sensor device, the data acquisition adapted to collect current and voltage output from the three-dimensional capacitance sensor; a processing system in communication with the data acquisition electronics, the processing system programmed with instructions for executing on the processing system to reconstruct a three-dimensional volume-image from the current and voltage output collected by the data acquisition electronics; wherein the three-dimensional capacitance sensor device is comprised of at least two planes of electrodes to provide sensor sensitivity in the axial and radial directions; wherein the processing system is programmed with an image reconstruction algorithm adapted to produce an image using capacitance data collected by the system and wherein the processing system is programmed with an image reconstruction algorithm adapted to produce an image using inductance data collected by the system.

The foregoing and other features and advantages of the present invention will be apparent from the following more detailed description of the particular embodiments, as illustrated in the accompanying drawings.

### BRIEF DESCRIPTION OF THE DRAWINGS

In addition to the features mentioned above, other aspects of the present invention will be readily apparent from the following descriptions of the drawings and exemplary embodiments, wherein like reference numerals across the several views refer to identical or equivalent features, and wherein:

FIG. 1 illustrates one embodiment of the Electro-Magneto Volume Tomography (EMVT) electrode and its accompanying circuitry for multi-modal function.

FIG. 2A illustrates an embodiment of the EMVT sensor electrode.

FIG. 2B illustrates another embodiment of the EMVT sensor electrode.

FIG. 3A illustrates another embodiment of a EMVT sensor electrode.

FIG. 3B illustrates another embodiment of an EMVT sensor electrode.

FIG. 3C illustrates another embodiment of an EMVT sensor electrode.

FIG. 3D illustrates another embodiment of an EMVT sensor electrode.

FIG. 4 illustrates an embodiment of the EMVT electrode wherein the coil configuration is arranged in a non-concentric way to give certain regions more or less magnetic field strength.

FIG. 5A illustrates one embodiment of a square EMVT sensor electrode.

FIG. 5B illustrates one embodiment of a circular EMVT sensor electrode.

FIG. 5C illustrates one embodiment of a triangular EMVT sensor electrode.

FIG. 6 illustrates the EMVT system circuitry when it is operating in Electrical Capacitance Tomography mode.

FIG. 7 illustrates the EMVT system circuitry when it is operating in Magnetic Induction Tomography mode.

FIG. 8 illustrates the EMVT system circuitry when it is operating in Magnetic Pressure Sensing mode.

FIG. 9 illustrates the EMVT system operating in adaptive mode.

FIG. 10 illustrates an embodiment of the EMVT sensor as a cylindrical shape around a cylindrical column.

FIG. 11 illustrates an embodiment of the EMVT sensor as a flat plane.

FIG. 12 illustrates embodiments of the EMVT sensor as partial and full enclosures of a volume.

### DETAILED DESCRIPTION OF EXEMPLARY EMBODIMENT(S)

The following detailed description of the example embodiments refers to the accompanying figures that form a part thereof. The detailed description provides explanations by way of exemplary embodiments. It is to be understood that other embodiments may be used having mechanical and electrical changes that incorporate the scope of the present invention without departing from the spirit of the invention.

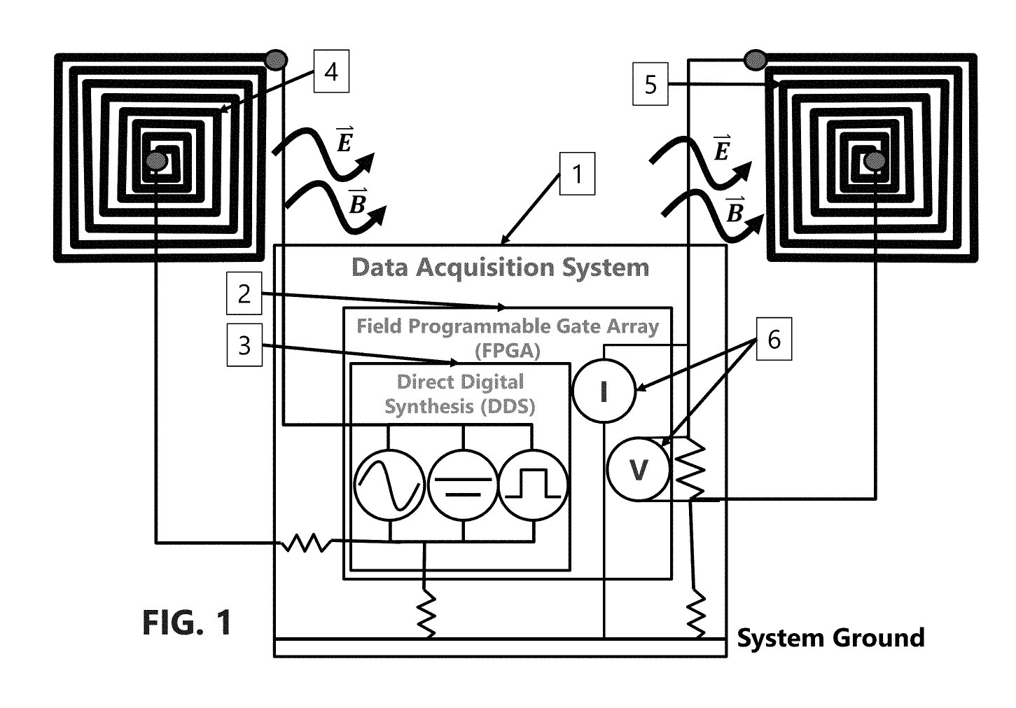

FIG. 1 illustrates one embodiment of the EMVT sensor electrodes, data acquisition system, and accompanying circuitry for multi-modal function. The data acquisition system (1) controls and measures multi-modal signals. The data acquisition system (DAS) is comprised of a microcontroller or Field Programmable Gate Array (2) (FPGA), which controls system operation and can be reprogrammed if necessary. Within this FPGA, there is a Direct Digital Synthesizer (DDS) block (3), which is able to generate and output different waveforms. These waveforms can be DC, sinusoidal AC, or different periodic signals. A single or multiple waveforms can be outputted by this component. The DDS can act as an AC power source, which means it can be an AC voltage source and an AC current source at the same time. By passing the AC voltage the DDS outputs through a resistor and closing the loop, an AC voltage is applied on the electrode and an AC current running through it at the same time.

Such waveforms are produced by the DDS (3), are outputted by the FPGA (2) and data acquisition system (1), and excite or activate one of the EMVT sensor electrodes (4) (i.e., the sending/driving electrode). This electrode then sends low-frequency, decoupled electric and magnetic waves through the sensing region. The electric and magnetic waves reach another electrode (5) and excite it. The electric field produces a voltage on the receiving electrode (5), and the magnetic field produces a current through the electrode. These voltages and currents are measured separately (6) but simultaneously within the data acquisition system and are sent to the FPGA (2) for data processing and recording (current meter designated at (6) by circled “I” and voltage meter designated at (6) by circled “V”). The data acquisition system preferably has one activation circuit and at least two measuring quantities, all preferably housed in the same enclosure. The voltage and current detected by the DAS is sent to another processing computer for imaging the sensed region of the sensor as previously incorporated with previous ECVT, AECVT and DCPT systems.

In this embodiment, the Data Acquisition System (DAS) is a component or circuit that activates an electric source (voltage or current source) on a sender element (e.g., electrode) and senses its effect on a receiver element (another electrode) by measuring an electric measurement (voltage or current effect). Also, the DAS preferably applies filters to condition the signal and typically converts it into a digital format (using analog to digital converters) to be fed to a computer or smart machine. The electric source is preferably a current or voltage source with a frequency, amplitude, and phase values. The measured signal is also a voltage or current signal with an amplitude, frequency, and phase information. It is appreciated that in another embodiment, the activation circuit portion and the receiver/data acquisition portion of the circuit can be separated into two separate components, circuits or instruments.

The image processing system receives information of the sender and receiver sensor formations and their order. This information is typically stored in what is called a sensitivity matrix. The matrix matches the response of each sender-receiver combination to a geometric location. When electric signals from all activations are received by the algorithm (this is typically called a frame of data); the algorithms uses the sensitivity matrix and the data frame to match the most probable distribution of material in the imaging domain. This processes of finding the most probable distribution can be a simple direct matching or more advanced optimization iterative algorithms. In this embodiment of the present invention, there would be two sensitivity matrixes; one for the capacitive effect and the other for the magnetic effect.

The system of FIG. 1 is adapted to combine and take measurements using multiple aforementioned noninvasive sensing techniques into an integrated unit in a unique fashion not previously known. This instrument can employ all or some of the following measurement techniques: Electrical Capacitance Tomography, Electrical Capacitance Volume Tomography, Adaptive Electrical Capacitance Volume Tomography, Displacement Current Volume Tomography, Magnetic Pressure Sensing, and Magnetic Inductance Tomography.

In the preferred embodiment, the electrodes used for a particular application using one measurement technique can be used for other measurement techniques. They react to both electric and magnetic fields. The coiling feature they have makes them generate or detect magnetic field and the surface area they have make them act in capacitive mode.

In one example, the driving electrode is connected to an AC power source. A resistor, connected in series between the AC power source the electrode, is used to generate an AC current through the electrode. A receiving electrode generates its own AC wave via electric and magnetic fields passing through the imaging domain. This generated wave causes AC current to flow through the isolation circuit and accompanying resistor (5) of the receiver electrode. All electrodes can act as either a driver or receiver, and are connected to a common data acquisition system (6).

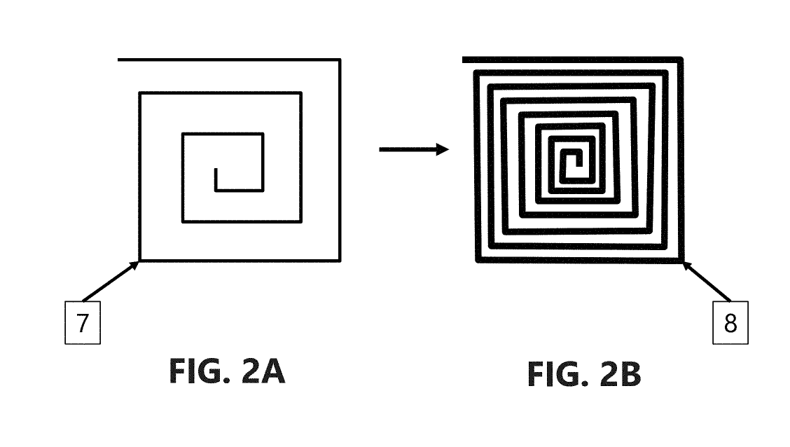

FIG. 2A illustrates an embodiment of the EMVT sensor electrode and FIG. 2B illustrates another embodiment of the EMVT sensor electrode. The electrodes can be configured with less (7) and more (8) turns within its coil, and a thinner (7) and thicker (8) conductor in the coil. By increasing the number of coils and the closeness of these coils, both the inductance and capacitance of the electrode are increased, respectively. By increasing the thickness of the conductor in the coil, the capacity of the electrode to carry current is increased. Horizontal surface area controls the capacitance feature of the sensors, and the number of coils controls the inductive feature.

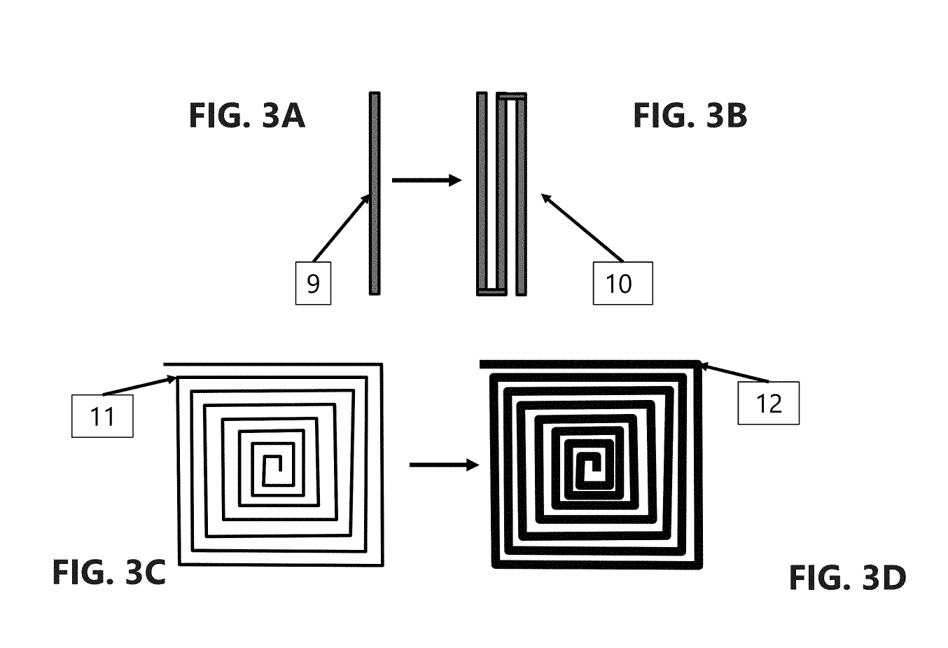

FIG. 3 illustrates four embodiments of the EMVT sensor electrode with varying conductor dimensions. FIG. 3A illustrates one embodiment of a EMVT sensor electrode, FIG. 3B illustrates another embodiment of an EMVT sensor electrode. By increasing the number of coil layers in the electrode in the orthogonal direction (9 and 10), the inductance of the conductor is increased while maintaining the same surface area. FIG. 3C illustrates another embodiment of an EMVT sensor electrode (11), FIG. 3D illustrates another embodiment of an EMVT sensor electrode. By increasing the width of the conductor along its plate surface (10 and 12), both the ampacity and capacity of the conductor are increased.

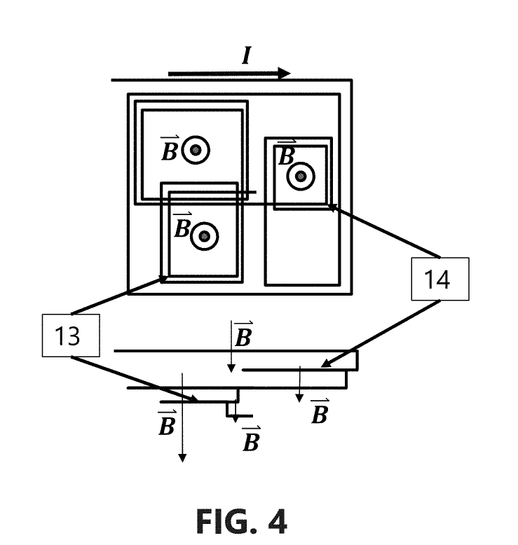

FIG. 4 illustrates an embodiment of the EMVT sensor electrode where the coiling configuration is not concentric, but instead localized coils (13 and 14) allow for finer control of magnetic field strength at certain positions and in certain directions, while maintaining the same overall capacity of the electrode when operating in capacitance plate mode.

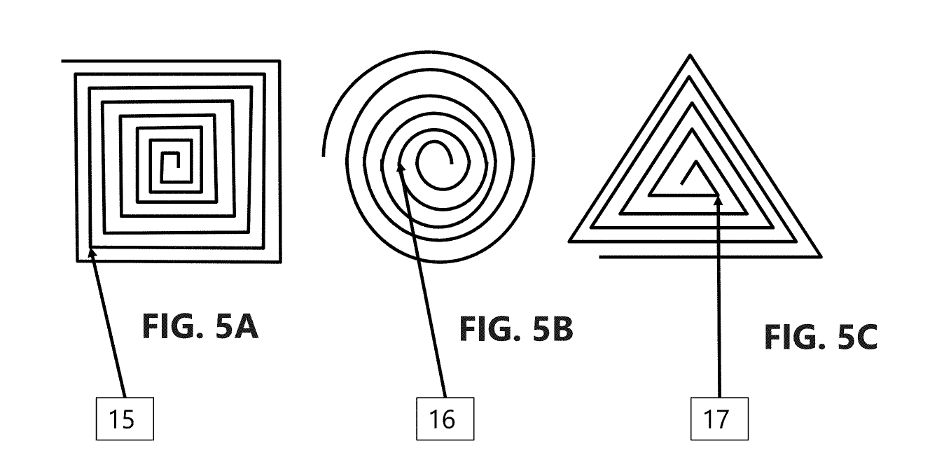

FIGS. 5A-C illustrate three embodiments of the EMVT sensor electrode as coils which are square (15), circular (16), and triangular (17). FIG. 5A illustrates one embodiment of a square EMVT sensor electrode. FIG. 5B illustrates one embodiment of a circular EMVT sensor electrode. FIG. 5C illustrates one embodiment of a triangular EMVT sensor electrode. Any polygonal or arbitrary coil shape is possible.

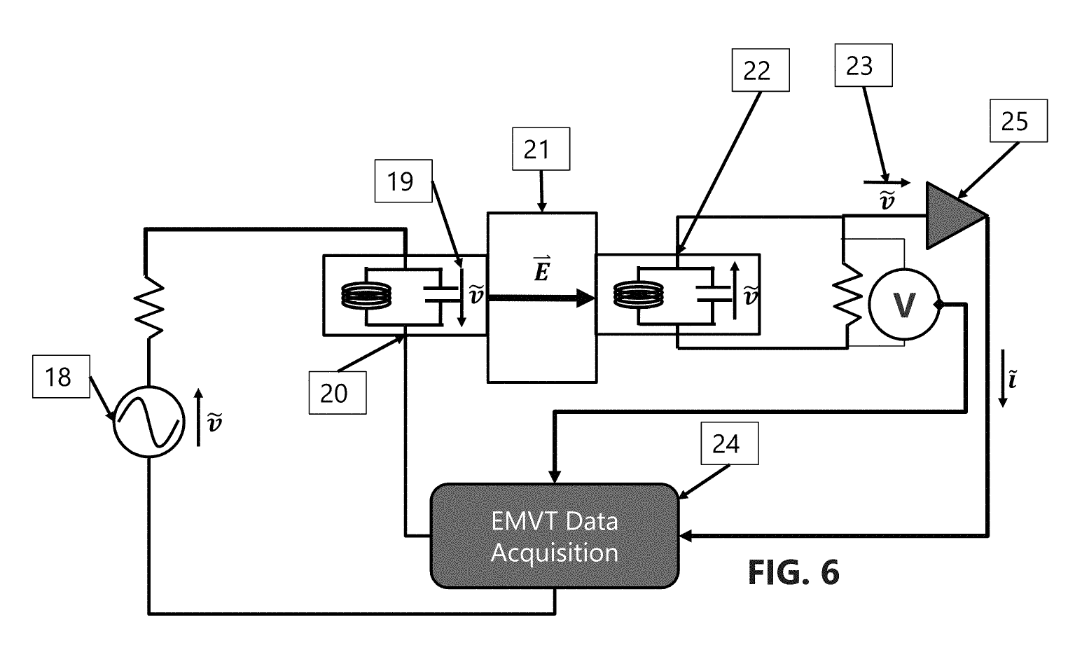

FIG. 6 illustrates an embodiment of the EMVT system and circuitry when operating in a capacitance plate mode. An AC power source (18) sends an AC voltage (19) to a driving electrode (20), here represented by a circuit having both an inductor and capacitor. An electric field propagates through the sensing region (21) and is detected by another electrode (22). The AC voltage (23) on the detecting electrode (22) is measured by the EMVT system electronics (24) as an AC current via converter circuitry (25).

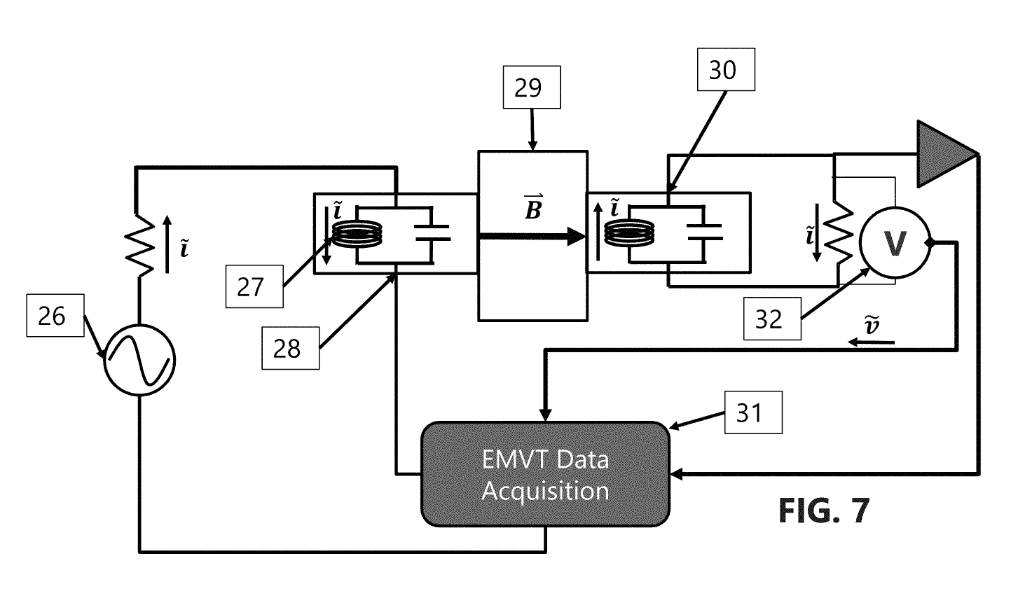

FIG. 7 illustrates an embodiment of the EMVT system and circuitry when operating in MIT mode. An AC power source (26) sends an AC current (27) through a driving electrode (28). A magnetic field propagates through the sensing region (29) and induces a current in another electrode (30). The AC current flowing through the detecting electrode (30) is measured by the EMVT system electronics (31) as an AC voltage via a voltmeter across a resistor (32).

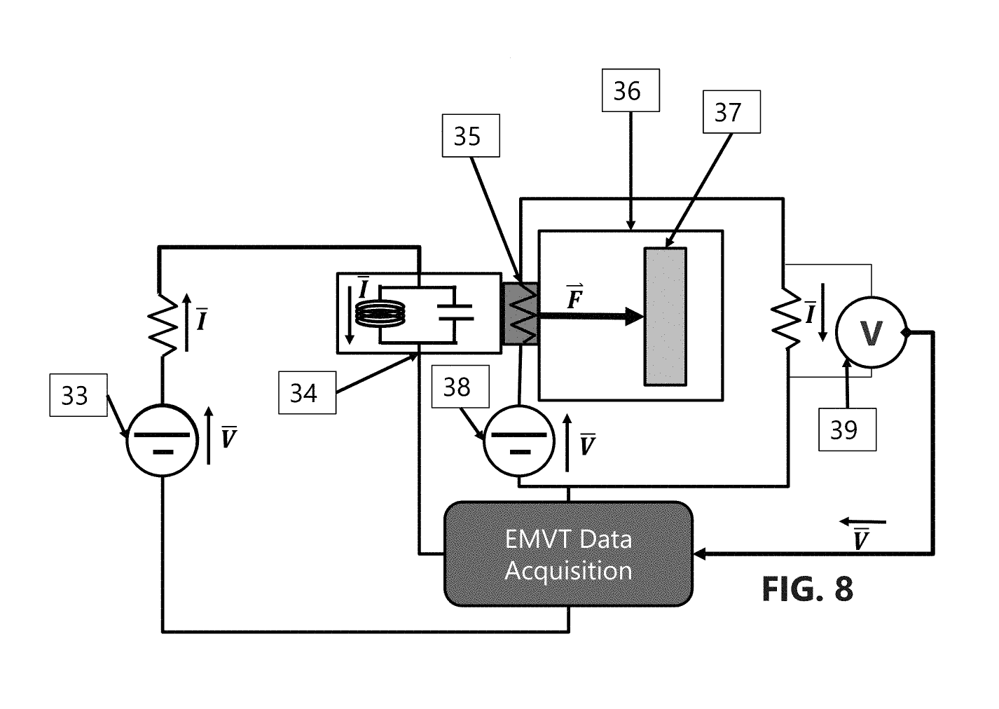

FIG. 8 illustrates an embodiment of the EMVT system and circuitry operating in Magnetic Pressure Sensing (MaPS) mode. A DC or AC power source (33) sends a current through an electrode (34). A non-magnetic force sensor (35) rests against this electrode (34). The electrode (34) generates a magnetic field that propagates into the sensing region (36). If magnetic, ferromagnetic, or ferromagnetic material (37) is present within the sensing region, the magnetic field will enact a force on the electrode (34). This force changes the resistance of the force sensor (35). The force sensor resistance is measured by running constant power (38) though the sensor and measuring the voltage across the device via a voltage divider circuit (39). The measured resistance is translated into a force magnitude, which is then related to quantity and location of magnetic, ferromagnetic, or ferromagnetic material within the sensing region (37).

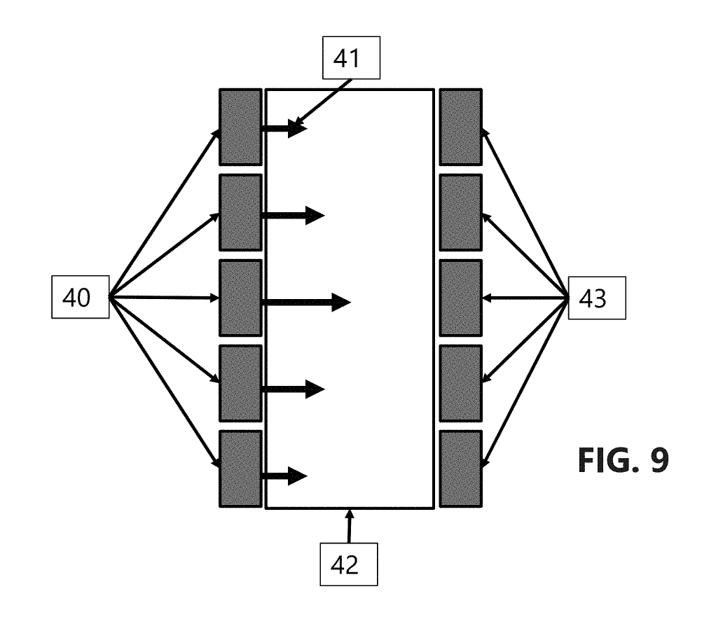

FIG. 9 illustrates an embodiment of the EMVT sensor operating in adaptive mode. Several electrodes (40) can be excited at the same time, though each electrode alone can be excited with its own unique excitation (41). Individual excitations can vary in terms of amplitude, phase shift, and frequency. These individual excitations enter the sensing region (42), and combine or negate one another to detect certain regions of the overall volume with more or less resolution. Similarly, multiple electrodes (43) can be treated as a singular detection electrode by collectively considering the detected signals at each receiver electrode (43). The aggregate response from all sensor combinations is used through image reconstruction techniques to reconstruction images of dielectric, conductive, or ferromagnetic material distribution.

In another embodiment, the DAS system is adapted to activate the electrodes for measuring current phases as used in other DCPT system. DCPT is detected the same way here as it is in previously discussed systems. For example, since an AC voltage is measured at the receiver electrode, the phase of that AC wave can be measured to get displacement current phase in DCPT-only systems. The same can be accomplished with an AC current. That is, the received induction current from MIT also has a phase shift which is measured.

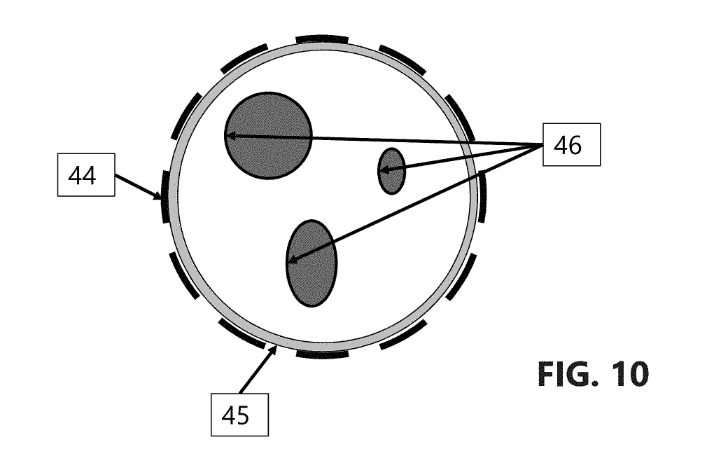

FIG. 10 illustrates an embodiment of the EMVT sensor as a cylindrical shell. The view is from the top of the cylindrical sensor, looking along its axis. An array of electrodes (44) is aligned along the surface of a cylindrical vessel (45). The EMVT sensor and system detects objects (46) within this vessel.

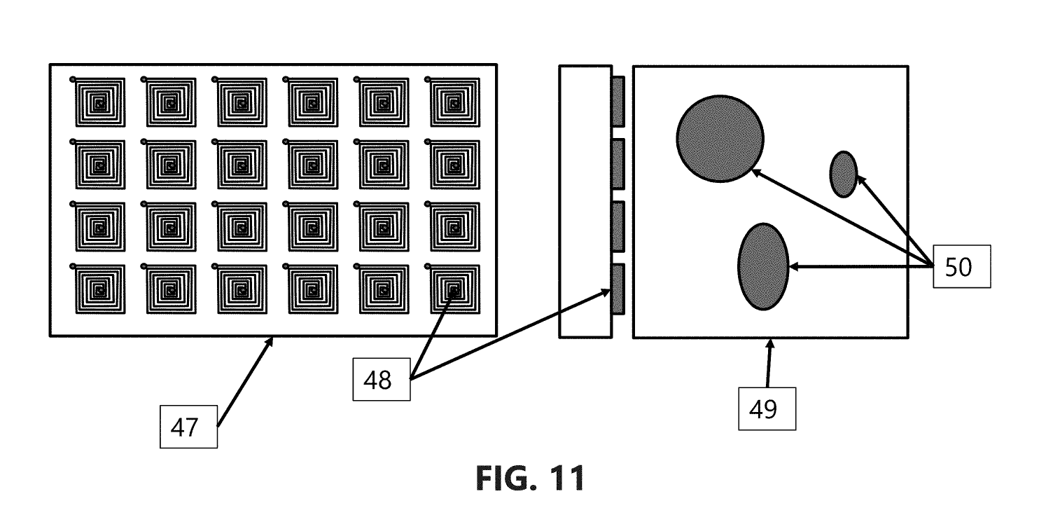

FIG. 11 illustrates an embodiment of the EMVT sensor as a flat plane. The views are from the front and side of the sensor (47). An array of electrodes (48) is aligned along the surface of the sensor. The EMVT sensor and system is pointed towards a wall, floor, ceiling, or space before it (49) and detects objects (50) within this region.

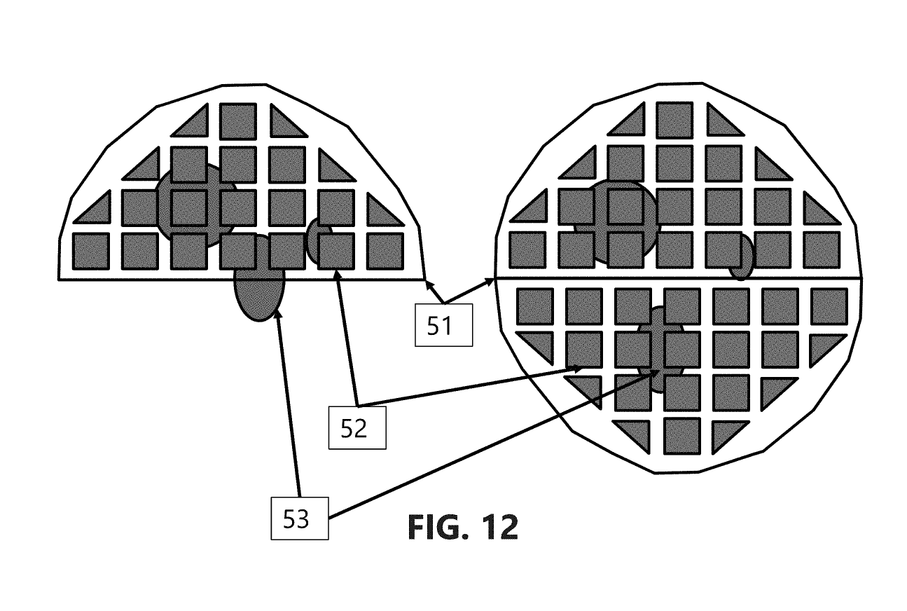

FIG. 12 illustrates embodiments of the EMVT sensor as a partial and full enclosure (51) of a volume. An array of electrodes (52) is aligned the surface of the sensor (51). The sensor detects objects (53) that are enclosed, partially enclosed, or nearby the sensing region.

While certain embodiments of the present invention are described in detail above, the scope of the invention is not to be considered limited by such disclosure, and modifications are possible without departing from the spirit of the invention as evidenced by the following claims.

What is claimed is:

1. A system for generating a tomograph of a vessel interior or other object, the system comprising:
a capacitance sensor comprising a plurality of electrodes for placement around the vessel or the object, wherein the capacitance sensor is adapted to provide electric field distribution and sensor sensitivity in three geometric dimensions;
an activation circuit for activating the capacitance sensor with an activation signal, and wherein the activation circuit is adapted to vary the activation signal by amplitude, phase, and frequency;
a data acquisition circuit in communication with the capacitance sensor for receiving output signals from the capacitance sensor, the data acquisition adapted to collect current and voltage output from the capacitance sensor; and
a processing system in communication with the data acquisition electronics, the processing system programmed with instructions for executing on the processing system to reconstruct a volume-image from the current or voltage output collected by the data acquisition electronics.

2. A system according to claim 1, wherein the activation signal is a sinusoidal AC signal.

3. A system according to claim 1, wherein the capacitance sensor is comprised of at least two planes of electrodes to provide sensor sensitivity in the axial and radial directions.

4. A system according to claim 1, wherein the processing system is programmed with an image reconstruction algorithm adapted to produce an image using capacitance data collected by the system.

5. A system according to claim 4, wherein the image reconstruction algorithm is adapted to provide real-time imaging of multiphase flow within the vessel.

6. A system according to claim 1, wherein the processing system is programmed with an image reconstruction algorithm adapted to produce an image using inductance data collected by the system.

7. A system according to claim 1, wherein the object is a human body.

8. A system according to claim 1, wherein the processing system is programmed with instructions to: 1) convert a three-dimensional image into an image vector, wherein elements of the image vector are voxels of the three-dimensional image; 2) define a three-dimensional sensitivity matrix related to the image vector and based on geometry of the geometrically capacitance sensor and a matrix of measured capacitance; 3) compute a volume image vector using a reconstruction algorithm selected based on the three-dimensional sensitivity matrix and matrix of the measured capacitance; and 4) convert the volume image vector to the three-dimensional volume-image.

9. A system according to claim 1, wherein the activation circuit is a direct digital synthesizer adapted to generate different types of signals.

10. A system according to claim 1, wherein the capacitance sensor is any shape or arrangement of electrodes that provides a three-dimensional electric field intensity in three directions with substantially equal strength.

11. A system according to claim 1, further comprising a strain gauge and wherein the activation circuit is adapted to apply a current though the capacitance sensor, and wherein the capacitance sensor is comprised of a first and second electrode, wherein the strain gauge is operationally placed against the first electrode, and wherein the first electrode acts as a magnet and is attracted to or repelled by any magnetic material inside the capacitance sensor, and wherein the system is adapted to measure a magnitude of force from the strain gauge.

12. A system according to claim 11, wherein the processing system is programmed with instructions for executing on the processing system to detect and image magnetic materials.

13. A system according to claim 1, wherein the system including the capacitance sensor is adapted to simultaneously measure variations in both capacitance and power corresponding to permittivity and conductivity distribution.

14. A system according to claim 1, further comprising:
a DC activation signal used as an excitation signal for the system.

15. A system according to claim 1, wherein a three-dimensional imaging domain of the capacitance sensor is divided into voxels and wherein the data acquisition electronics receives data for each voxel and wherein the processing system is programmed with instructions for executing on the processing system for reconstructing the three-dimensional volume-image based on the data received for each voxel.

16. A system according to claim 1, wherein the system is adapted to distribute electric field intensity or sensor sensitivity substantially equally within the capacitance sensor.

17. A system according to claim 1, wherein the processing system is programmed with instructions for executing on the processing system for providing a multi-criterion optimization based image reconstruction technique.

18. A system according to claim 1, wherein the system including the capacitance sensor is adapted to obtain both capacitance and impedance flow information.

19. A system according to claim 1, wherein there are N number of electrodes and wherein the system is programmed to collect N(N−1)/2 capacitance measurements for all of the combinations of electrode pairs for use in volume-image reconstruction, and wherein the capacitance sensor is comprised of at least two planes of electrodes in the axial direction to provide sensor sensitivity in the radial and axial directions.

20. A system according to claim 1 wherein the system provides substantially equal sensor sensitivity over the entire sensing domain of the capacitance sensor.

21. A system according to claim 1, wherein the capacitance sensor is comprised of at least two rows or planes of electrodes to provide sensor sensitivity in the radial and axial directions.

22. A system according to claim 21, wherein each of the plurality of electrodes are connected to a channel of the data acquisition circuit and wherein there are an N number of electrodes and the system is adapted to take N(N−1)/2 capacitance measurements for each electrode pair.

23. A system according to claim 22, wherein the processing system is programmed with instructions for executing on the processing system to reconstruct the three-dimensional volume-image from the actual capacitance measurements collected by the data acquisition electronics without the need for averaging.

24. A system according to claim 23, wherein the system provides substantially equal sensitivity variation over the sensing domain of the capacitance sensor.

25. A system according to claim 21, wherein a three-dimensional imaging domain of the capacitance sensor is divided into voxels and wherein the data acquisition electronics receives data for each voxel and wherein the processing system is programmed with instructions for executing on the processing system for reconstructing the three-dimensional volume-image based on the data received for each voxel.

26. A system according to claim 21, wherein the arrangement of the plurality of electrodes or the shape of the plurality of electrodes can be changed to vary the sensor sensitivity.

27. A system according to claim 21, wherein a sensitivity matrix of the capacitance sensor has a dimension of (M×N), where M is the number of electrode pair combinations and N is the number of voxels.

28. A system according to claim 21, wherein the system is adapted to distribute electric field intensity or sensor sensitivity substantially equally within the capacitance sensor.

29. A system according to claim 21, wherein the capacitance sensor is adapted to provide interrogation of the whole volume of an imaging domain of the capacitance sensor and wherein the processing system is programmed with instructions for executing on the processing system for reconstructing the three-dimensional volume-image of the vessel interior or other object based on the interrogation of the whole volume of the imaging domain.

30. A system according to claim 1, wherein the capacitance sensor is comprised of a plurality of electrodes comprised of a non-magnetic conductor formed by a coil of wire.

31. A system according to claim 1, wherein the capacitance sensor is comprised of a plurality of electrodes wherein each of the electrodes are formed from a coil of wire and wherein the system is further comprised of a ferromagnetic material placed in a center of the at least one of the electrodes.

32. A system according to claim 1, wherein the capacitance sensor is comprised of a plurality of electrodes wherein each of the electrodes are formed from a coil of wire into a concentric shape.

33. A system according to claim 1, wherein the data activation circuit is adapted to measure the phase of the current output and wherein the processing system is programmed with instructions for executing on the processing system to reconstruct an image of material within the capacitance sensor using the phase of the current output.

34. A system for generating a three-dimensional tomograph of a vessel interior or other object, the system comprising:
a capacitance sensor comprising a plurality of electrodes for placement around the vessel or the object, wherein the capacitance sensor is adapted to provide electric field distribution and sensor sensitivity in three geometric dimensions;
an activation circuit for activating the capacitance sensor with an activation signal; wherein the activation circuit is adapted to vary the activation signal by amplitude, phase, and frequency;
a data acquisition circuit in communication with the capacitance sensor for receiving output signals from the capacitance sensor, the data acquisition adapted to collect current and voltage output from the capacitance sensor;
a processing system in communication with the data acquisition electronics, the processing system programmed with instructions for executing on the processing system to reconstruct a three-dimensional volume-image from the current and voltage output collected by the data acquisition electronics;
wherein the capacitance sensor is comprised of at least two planes of electrodes to provide sensor sensitivity in the axial and radial directions; and
wherein the processing system is programmed with an image reconstruction algorithm adapted to produce an image using capacitance data collected by the system and wherein the processing system is programmed with an image reconstruction algorithm adapted to produce an image using inductance data collected by the system.

35. A system according to claim 34, wherein the activation signal is a square wave.

36. A system according to claim 34, wherein the processing system is programmed with instructions to: 1) convert a three-dimensional image into an image vector, wherein elements of the image vector are voxels of the three-dimensional image; 2) define a three-dimensional sensitivity matrix related to the image vector and based on geometry of the geometrically capacitance sensor and a matrix of measured capacitance; 3) compute a volume image vector using a reconstruction algorithm selected based on the three-dimensional sensitivity matrix and matrix of the measured capacitance; and 4) convert the volume image vector to the three-dimensional volume-image.

37. A system according to claim 34, the activation circuit is a direct digital synthesizer adapted to generate different types of signals.

38. A system according to claim 1, further comprising a strain gauge and wherein the activation circuit is adapted to apply a current though the capacitance sensor, and wherein the capacitance sensor is comprised of at least two electrodes, wherein the strain gauge is operationally placed against a first electrode, and wherein the first electrode acts as a magnet and is attracted to or repelled by any magnetic material inside the capacitance sensor, and wherein the system is adapted to measure a magnitude of force from the strain gauge.

39. A system according to claim 11, wherein the processing system is programmed with instructions for executing on the processing system to detect and image magnetic materials.

40. A system according to claim 34, wherein the data activation circuit is adapted to measure the phase of the current output and wherein the processing system is programmed with instructions for executing on the processing system to reconstruct an image of material within the capacitance sensor using the phase of the current output.

41. A system for generating a three-dimensional tomograph of a vessel interior or other object, the system comprising:
a capacitance sensor comprising a plurality of electrodes for placement around the vessel or the object, wherein the capacitance sensor is adapted to provide electric field distribution and sensor sensitivity in three geometric dimensions;
an activation circuit for activating the capacitance sensor with an activation signal; wherein the activation circuit is adapted to vary the activation signal by amplitude, phase, and frequency;
a data acquisition circuit in communication with the capacitance sensor for receiving output signals from the capacitance sensor, the data acquisition adapted to collect current and voltage output from the capacitance sensor;
a processing system in communication with the data acquisition electronics, the processing system programmed with instructions for executing on the processing system to reconstruct a three-dimensional volume-image from the current and voltage output collected by the data acquisition electronics;
wherein the capacitance sensor is comprised of at least two planes of electrodes to provide sensor sensitivity in the axial and radial directions;
wherein the processing system is programmed with an image reconstruction algorithm adapted to produce an image using capacitance data collected by the system and wherein the processing system is programmed with an image reconstruction algorithm adapted to produce an image using inductance data collected by the system; and
wherein the system is further comprised of a strain gauge and wherein the activation circuit is adapted to apply a current though the capacitance sensor, and wherein the capacitance sensor is comprised of a first electrode, and wherein the strain gauge is operationally placed against the first electrode, and wherein the first electrode acts as a magnet and is attracted to or repelled by any magnetic material inside the capacitance sensor, and wherein the system is adapted to measure a magnitude of force from the strain gauge.

42. A system according to claim 41, wherein the processing system is programmed with instructions to: 1) convert a three-dimensional image into an image vector, wherein elements of the image vector are voxels of the three-dimensional image; 2) define a three-dimensional sensitivity matrix related to the image vector and based on geometry of the geometrically capacitance sensor and a matrix of measured capacitance; 3) compute a volume image vector using a reconstruction algorithm selected based on the three-dimensional sensitivity matrix and matrix of the measured capacitance; and 4) convert the volume image vector to the three-dimensional volume-image.

43. A system according to claim 41, wherein the activation circuit is a direct digital synthesizer adapted to generate different types of signals.

44. A system according to claim 41, wherein the processing system is programmed with instructions for executing on the processing system to detect and image magnetic materials.

45. A system according to claim 41, wherein the data activation circuit is adapted to measure the phase of the current output and wherein the processing system is programmed with instructions for executing on the processing system to reconstruct an image of material within the capacitance sensor using the phase of the current output.

46. A system according to claim 1, where inductance is controlled by the number of coils in the capacitance sensor and the capacitance by a frontal surface area of the capacitance sensor.

47. A system according to claim 1, where the capacitance sensor can operate in multimodal mode and perform electrical capacitance volume tomography and electrical magnetic volume tomography.

48. A system according to claim 1, where the capacitance sensor can provide a control over magnetic field variation by using multiple axis points of coiling.

49. A system according to claim 1, wherein the capacitance sensor is of a cylindrical shape to close around a cylindrical column.

50. A system according to claim 1, wherein capacitance sensor is of a flat planar shape to scan into a flat body such as a floor, wall, and ceiling.

51. A system according to claim 34, wherein the electrodes are each comprised of a non-magnetic conductor spun into a coil of a single layer or multiple layers.

52. A system according to claim 34, wherein each of the electrodes are spun in a coil wherein the coil is square in shape.

53. A system according to claim 34, wherein each of the electrodes are spun in a coil and wherein the coils are circular in shape.

54. A system according to claim 34, wherein at least one electrode is comprised of small sub-coils within the electrode that concentrate magnetic fields within the sub-coils.

55. A system according to claim 34, wherein at least one electrode is comprised of a conductor that extends in a direction away from its surface to increase its cross-section and thus its ampacity.

56. A system according to claim 34, wherein at least one electrode is comprised of a conductor that is widened to increase its cross-section and thus ampacity and capacity for charge collection.

57. A system according to claim 34, wherein at least one electrode is comprised of wound coils and wherein the number of coils is varied to increase or decrease inductance and capacitance of the electrode.
